(19) United States
(12) Patent Application Publication
(10) Pub. No.: US 2004/0115259 A1
(43) Pub. Date: Jun. 17, 2004

(54) Method for producing a floating tablet containing an active principle and the tablet obtained

(76) Inventors: Frederique Bordes, Bordeaux (FR); Sylvie Cuart, Pessac (FR); Laurent Terrassin, Le Bouscat (FR)

Correspondence Address: YOUNG & THOMPSON, 745 SOUTH 23RD STREET 2ND FLOOR, ARLINGTON, VA, 22202

(21) Appl. No.: 10/467,410

(22) PCT Filed: Feb. 7, 2002
(86) PCT No.: PCT/FR02/00474

(30) Foreign Application Priority Data
Feb. 8, 2001 (FR) 01/01711
Dec. 21, 2001 (FR) 01/16705

(51) Int. Cl. A61K 9/20; B27N 3/00

(52) U.S. Cl. 424/465; 264/109

(57) ABSTRACT

Method for producing a tablet containing alfuzosin, which comprises the following steps: a given quantity of alfuzosin is prepared in accordance with the dosage for a given dissolution time; the quantity of active principle is homogeneously mixed with a quantity of carrier of between 50 and 99.9% of the total weight, the carrier being chosen from among at least one compound from the family of cellulose derivatives and/or povidone derivatives and/or polyvinyl acetate derivatives; the mixture is compressed with a force in order to produce a homogeneous monolithic tablet that floats immediately, in the gastric medium. The invention also covers the tablet obtained.

The present invention relates to a process for the production of a floating tablet including as active principle alfuzosin. The invention also covers the tablet thus obtained.

It is known that it is useful for certain active principles to undergo controlled release so as to limit the number of doses whilst ensuring optimum effectiveness. Thus, the administration of oral forms with immediate release requires several administrations per day which, in addition to the inconvenience that arises, can disturb the observance of the treatment.

As concerns more particularly alfuzosin hydrochloride, used particularly in the treatment of benign hypertrophy of the prostate and in the treatment of premature ejaculation, the controlled release is all the more necessary.

This molecule, with a short half-life of elimination, has the characteristic of being preferentially absorbed in the upper portion of the gastrointestinal tract, more particularly in the duodenum and the jejunum.

In U.S. Pat. No. 6,149,940, there is proposed a compound adapted for the oral route seeking to liberate alfuzosin hydrochloride. This tablet is constituted by at least two layers, which comprise:

the first, a mixture of hydrophilic polymers, 5.00 to 90.00%, and

the second, a mixture of hydrophilic polymers and alfuzosin hydrochloride. This second layer is compressed in a suitable manner and comprises auxiliary substances such that the alfuzosin hydrochloride will be released over a predetermined interval of time.

It has been proposed to control this release by addition of a third layer constituted also of hydrophilic polymers so as to protect the layer containing the alfuzosin hydrochloride and to slow the release, at least at the outset.

Such tablet has drawbacks. Thus, the tablet is initially heavier than the density of the gastric fluids in which it will be immersed. A risk is that the tablet will be expelled before having acquired sufficient flotation power. The kinetics of swelling of hydrophilic polymers is slow, of the order of a half hour, and this delay is sufficient that they could be expelled. A sufficient swelling which would avoid passage through the pylorum is achieved several hours after ingestion so that the dimensional characteristics and eventually mechanical retention cannot overcome the problem of lack of initial flotation.

The gastric dwell time also depends on the anatomy of the individual, the size of the pylorum, the rhythm of opening and closing, so that all these considerations give rise to wide variations.

As a result, controlled release under such conditions is difficult.

French patent application 2 784 583 discloses a multi-layer tablet, improved in that it comprises an effervescent couple which generates carbon dioxide in contact with gastric fluids. These bubbles of carbon dioxide confer flotation properties to this tablet, which permit it to remain in the stomach in a less haphazard way.

This flotation is completed by the presence of hydrophilic polymers which swell and increase the volume of the tablet over time.

Nevertheless, such a tablet remains complicated to produce industrially because it is necessary to work in an atmosphere of controlled humidity because of the effervescent couple. This also requires precautions to store the obtained products, given the presence of this effervescent couple, so that production is of higher cost.

The effervescent couple used contains sodium whose content is not desirable particularly in the case of a low sodium diet, which is another drawback.

The process of production according to the present invention, of a tablet containing alfuzosin, permits guaranteeing optimum dwell time in the stomach of the individual by immediate flotation, a controlled release following a profile whose kinetics can vary and be adapted particularly between kinetics of the order of 0 and 1, and to complete retention by flotation thanks to swelling. Moreover, the tablet obtained by the process according to the present invention can be produced at reduced cost and does not require sensitive preservation conditions.

To this end, the process of production according to the invention, of a tablet containing alfuzosin, is characterized in that it comprises the following steps:

preparation of a given quantity of alfuzosin as a function of the dosage for a given duration of dissolution,

homogeneous mixing of this quantity of active principle with a quantity of excipient from 50.00 to 99.90% of the total mass, the excipient being selected from at least one compound of the family of cellulosic derivatives and/or povidon derivatives and/or derivatives of polyvinyl acetate, and

compression of this mixture with a force permitting obtaining a monolithic homogeneous tablet with immediate flotation in the gastric medium.

Preferably, the excipient is present in the amount of 70.00 to 99.00% of the total mass of the tablet.

The alfuzosin is in the form of alfuzosin hydrochloride.

The compression is a direct compression.

To improve the homogeneity of the mixture and the compressibility of the grains, the compression is preceded by granulation in a moist or dry medium.

To obtain an order comprised between 0 and 1 of the given duration of dissolution, there are added kinetic modulators of the release of the alfuzosin hydrochloride.

As a compound of the family of cellulosic derivatives, can be considered more particularly hydroxypropylmethylcellulose and, after graduation, there is added this hydroxypropylmethylcellulose in an external phase.

The alfuzosin hydrochloride is introduced in the amount of 1 to 100 mg and more particularly 2.5, 5 or 10 mg.

The tablet has a weight comprised between 50 and 800 mg, preferably between 200 and 400 mg.

The invention also covers the monolithic tablet obtained, comprising alfuzosin hydrochloride.

The process of production according to the present invention is described hereafter and the obtained tablet is simultaneously characterized.

FIG. 1 shows the table of flotation of a tablet obtained by the process, with variations of the compressive force in three lots (A-C).

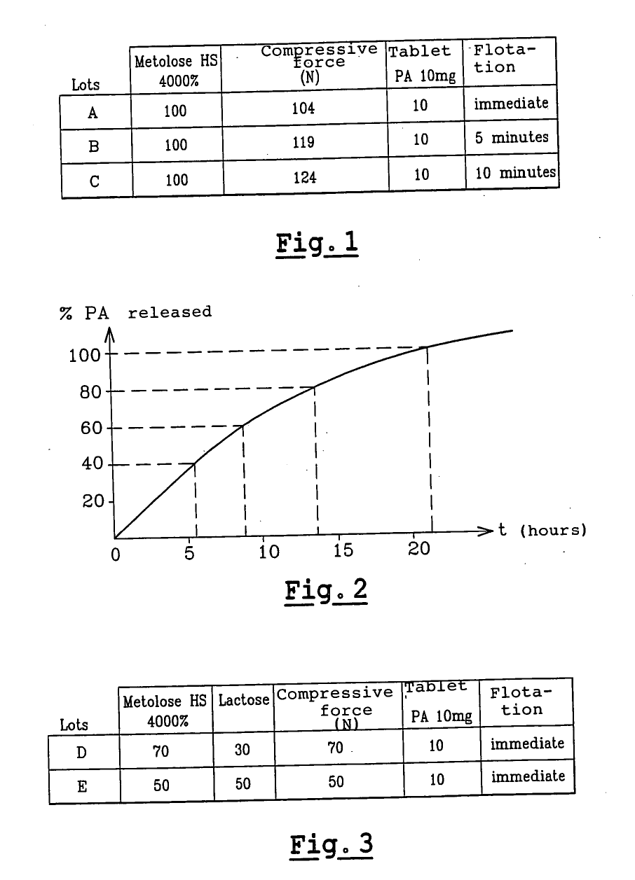

FIG. 2 shows the curve of dissolution obtained with the tablet of Lot A of the compositions of the table of FIG. 1,

FIG. 3 is a table showing flotation of a tablet with the inclusion of lactose, in two lots (D, E).

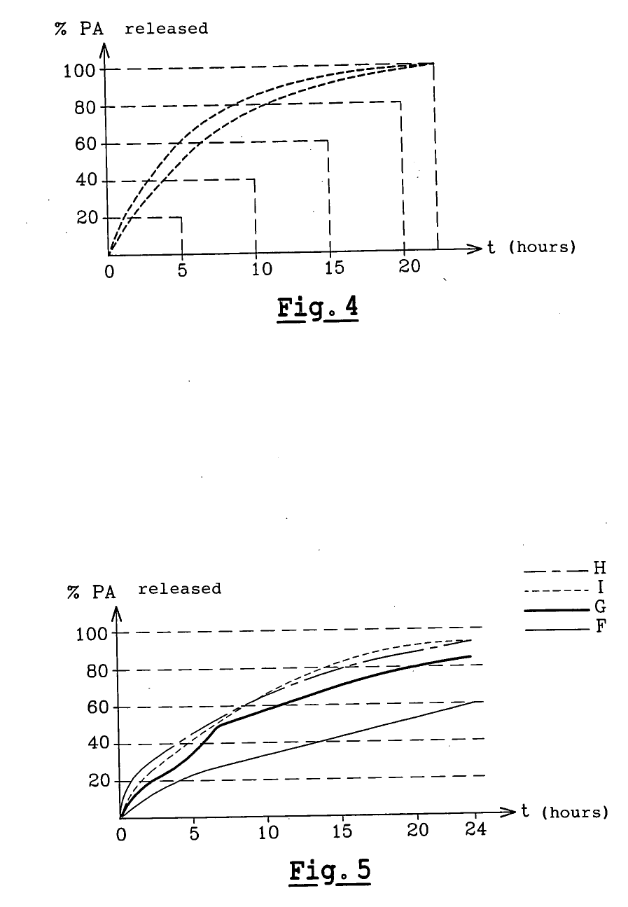

FIG. 4 shows two curves of dissolution in which the kinetics of release are modified.

FIG. 5 shows four curves of dissolution obtained with compositions F to I including as the basic excipient a derivative of polyvinyl acetate.

According to the invention, the process of production of a tablet based on alfuzosin and in the examples which follow alfuzosin hydrochloride, consists in compressing directly the powder or in compressing the granules obtained by humid or dry granulation. The mixture of alfuzosin hydrochloride and a low density compound permits producing a monolithic tablet, which will be homogeneous, such that the density of this tablet will be less than that of the gastric fluids.

The compressive force must be suitable to respect the density of the monolithic tablet, which will be homogeneous, which must remain less than that of the gastric fluids.

### EXAMPLE 1

There is prepared a mixture of alfuzosin hydrochloride 10 mg and a hydroxypropylmethylcellulose derivative or HPMC. There can be used as the hydroxypropylmethylcellulose derivative, the commercial product known by the trade name Metolose 90SH, with a viscosity of 4000 centipoises. The mixture is compressed directly. The tablet has a final weight of 400 mg.

The table of FIG. 1 shows that flotation is immediate for the composition compressed at 104 N.

It will be seen that if the force of compression is modified with an increase of the exerted force, the density becomes too great to ensure immediate flotation. The force of compression of the tablet should thus be maintained, as a function of its composition, at values such that the tablet floats immediately. Simple tests in the laboratory permit determining the useful values in each case.

In FIG. 2 is shown the profile of dissolution of alfuzosin hydrochloride (percentage of active principle or PA released as a function of time t in hours) under conventional test conditions for European pharmacology, namely, apparatus with paddles, 37° C., 100 turns per minute and in an acid medium of pH2, the dosage being obtained by spectrophotometric measurement at 254 nm.

There will be seen a kinetics of dissolution which decreases with time, corresponding substantially to kinetics of the order of 1 per 12 to 24 hours, which should be sought.

### EXAMPLE 2

There is substituted for a fraction of the hydroxypropylmethylcellulose derivative (Metolose 90SH, 4000 centipoises), a disintegrating agent, namely lactose.

The compression is direct compression. The values are determined by known laboratory tests.

The table of FIG. 3 shows that the flotation is immediate for applied compressive forces of 50 to 70 N, if the lactose is present at 30 or 50%.

On the curve of FIG. 4 (percentage of Active Principles released as a function of time t in hours), there will be noted that naturally the more the disintegrating agent is present, the less the kinetics of dissolution are linear.

The quantity of lactose should be adjusted as a function of the desired profile of dissolution.

### EXAMPLE 3

There is produced a tablet as in Example 1 but by choosing another derivative of hydroxypropylmethylcellulose. There is prepared a mixture of alfuzosin hydrochloride,10 mg, and there is used as derivative that sold under the same name Metolose 90SH, but with a viscosity of 15,000 centipoises. The tablet has a final identical weight of 400 mg. The force of compression is 82 N. The compression is direct compression determined from known laboratory tests.

The flotation is immediate.

The curve of dissolution is not affected by the choice of viscosity.

### EXAMPLE 4

In this test, the tablet of FIG. 1 is used, but the total weight of the compound is limited to 200 mg, with the same force of direct compression. It is again seen that the tablet has immediate flotation. The curve of dissolution is not changed.

### EXAMPLE 5

Prior to its compression, the active principle is suspended in a suitable solvent and granulated by a retardant polymer belonging to the family of polymethacrylates (particularly the one sold commercially under the name Eudragit RS 30 D).

The active principle is dried and then passed through a grill of 0.8 mm.

There is added as external phase a derivative of hydroxypropylmethylcellulose and the mixture thus obtained is compressed. There is obtained immediate flotation. The profile of release shows a slowing over 24 hours as indicated in FIG. 2. By slowing the kinetics, a tendency toward constant speed of release could be obtained, which is to say a more linear profile.

### EXAMPLE 6

A test is now carried out with recourse to a derivative of polyvinyl acetate sold under the name Kollidon SR. This commercial derivative comprises of the order of 80% polyvinyl acetate and 19% povidone and 1% stabilizer.

With this excipient, direct compression is facilitated and the recourse to a diluent is not necessary.

In this case, the four following compositions are tested:

F: 100% Kollidon SR

G: 90% Kollidon SR and 10% lactose (disintegrating agent)

H: 80% Kollidon SR and 20% lactose (disintegrating agent)

I: 70% Kollidon SR and 30% lactose (disintegrating agent),

which gives the curves of FIG. 5 which show that the profile can be adjusted so as to obtain kinetics of the order of 0 or 1 over 12 to 24 hours.

### EXAMPLE 7

A test is then carried out with a step of granulation prior to compression.

There is prepared a granulation solution in aqueous medium in which is dissolved the active principle, in this example at a level of 20%.

There is produced a mixture of 90% Kollidon SR and 10% of pre-gelatinized starch sold under the name Sepistab ST200.

There is carried out the step of granulation by spraying the wetting solution described above. The grains are then dried in a bed fluidized with air to obtain a water content less than 2%. The grains are then calibrated and lubrication is carried out in the external phase before compression.

There are obtained tablets whose kinetics of dissolution are substantially identical to those of Examples 1 and 2.

On the other -hand, the step of granulation permits improving the homogeneity of the mixture and the compressibility of the granules.

It will be seen that the process permits obtaining a tablet which is monolithic and homogeneous, with immediate flotation and with a substantially regular release of active principle.

The dosage of the active principle can be comprised between 1 and 100 mg and more particularly 2.5, 5 and 10 mg.

The weight of the tablet obtained is preferably comprised between 50 and 800 mg, more particularly between 200 and 400 mg.

The hydroxypropylmethylcellulose or its derivatives and/or the derivatives of povidone and of polyvinyl acetate are present in the amount of 50.00 to 99.00% of the total mass of the tablet.

A disintegrating agent can be added in the amount of 1to 50% by weight.

Other excipients can be added to modify the kinetics of release, such as clay, cross-linked polyvinylpyrrolidone, or certain cellulosic derivatives in small proportions such as hydroxypropylcellulose, cross-linked carbomethylcellulose, or else compounds of the family of N-acetylglucosamine, particularly chitosan.

Examples of agents permitting accelerating the kinetics of dissolution are hydrophilic agents added in the external phase: mannitol, lactose, starch and modified starch, sorbitol, xylitol or microcrystalline cellulose.

Examples of liposoluble diluant agents permit on the contrary slowing the release of the active principle, such as: palmitates, castor oil, glycerol monostearate. These agents can help render the obtained tablet even more buoyant by a decrease of the density of the system.

The kinetics of liberation can also be slowed by ensuring granulation of the active principle with retardation agents such as polyvinylpyrrolidone, gelatin, ethylcellulose, derivatives of polymethacrylates, alginic acid and its salts. The grains thus obtained are then mixed with hydroxypropylmethylcellulose, added in the external phase.

To practice the steps of the process, it may be necessary to add during compression lubricants such as: glycerol monostrearate, derivatives of polyoxyethylene glycol, glycerol behenate as well as flow agents such as derivatives of colloidal silica.

It will be understood that the simplified process of production permits reducing costs greatly. Not only the steps are fewer, but the conditions of execution are less complicated, because particularly of the absence of an effervescent couple.

Moreover, the kinetics of dissolution of the alfuzosin hydrochloride can be adjusted at least between an order of 0 and 1.

There is also noted a complementary effect arising from the nature of the constituents used, which is swelling. This is to say that the immediate flotation capacities are reinforced over time, because the weight decreases and the volume increases. Moreover, the risk of destratification of the layers of the existing tablets is avoided because the monolithic character gives rise to good mechanical strength.

It will be seen that the proportion of excipient is very important relative to that of the active principle. It was not obvious that such a ratio could lead to immediate flotation and that it could lead to control of the release profiles as has been indicated above.

1. Process for the production of a tablet containing alfuzosin, characterized in that it comprises the following steps:
preparation of a given quantity of alfuzosin as a function of the dosage for a predetermined duration of dissolution,
homogeneous mixing of this quantity of active principle with a quantity of excipient from 50.00 to 99.90% of the total mass, the excipient being selected from at least one compound of the family of cellulosic derivatives and/or derivatives of povidone and/or derivatives of polyvinyl acetate, and
compression of this mixture with a force permitting obtaining a monolithic homogenous tablet with immediate flotation in the gastric medium.

2. Process for production according to claim 1, characterized in that the excipient is preferably added in the amount of 70.00 to 99.00% of the total weight of the tablet.

3. Process for production according to claim 1 or 2, characterized in that the compression exerted is a direct compression after mixing of the active principle with the excipients.

4. Process for production according to claim 1 or 2, characterized in that, prior to compression, there is carried out a wet or dry granulation.

5. Process for production according to any one of the preceding claims, characterized in that the alfuzosin is in the form of alfuzosin hydrochloride.

6. Process for production according to any one of the preceding claims, characterized in that there are added modulators of the kinetics of release of the alfuzosin hydrochloride to obtain an order comprised between 0 and 1 of the given duration of dissolution.

7. Process for production according to any one of the preceding claims, characterized in that there are added modulators of the kinetics of release, such as clay, cross-linked polyvinylpyrrolidone, cellulosic derivatives in low proportions of the hydroxypropylcellulose, or cross-linked carbomethylcellulose or else compounds of the family of N-acetylglucosamine particularly chitosan.

8. Process for production according to any one of the preceding claims, characterized in that there are added one or more hydrophilic agents for accelerating release, selected from mannitol, lactose, starch or modified starch, sorbitol, xylitol or microcrystalline cellulose.

9. Process for production according to any one of claims 1 to 7, characterized in that there are added one or more agents for slowing release with decrease of density, selected from palmitates, castor oil, glycerol monostearate.

10. Process for production according to any one of the preceding claims, characterized in that the granulation of the active principle is ensured with retardation agents selected from polyvinylpyrrolidone, gelatin, ethylcellulose, polymethacrylate derivatives, alginic acid and it salts.

11. Process for production according to any one of the preceding claims, characterized in that there are used as compounds of the family of cellulosic derivatives, hydroxypropylmethylcellulose.

12. Process for production according to claim 10 and 11, characterized in that, after granulation, there is added hydroxpropylmethylcellulose in the external phase.

13. Process for production according to any one of the preceding claims, characterized in that there is added, during compression, at least one lubricant selected from glycerol monostearate, polyethylene glycol derivatives, glycerol behanate and/or a flow agent such as colloidal silica derivatives.

14. Process for production according to any one of the preceding claims, characterized in that the alfuzosin hydrochloride is introduced in an amount of 1 to 100 mg and more particularly 2.5, 5 or 10 mg.

15. Process for production according to any one of the preceding claims, characterized in that there is prepared a tablet whose weight is comprised between 50 and 800 mg, preferably between 200 and 400 mg.

16. Tablet comprising alfuzosin hydrochloride obtained by the practice of the process according to any one of claims 1 to 13.
